(19) United States
(12) Patent Application Publication
(10) Pub. No.: US 2010/0303922 A1
(43) Pub. Date: Dec. 2, 2010

(54) METHOD FOR THE PREPARATION OF BIOCOMPATIBLE POLYMERIC NANOPARTICLES FOR DRUG DELIVERY AND NANOPARTICLES PREPARED THEREBY

(75) Inventors: Soon Hong Yuk, Daejeon (KR); Keun Sang Oh, Daejeon (KR); Won Tae Jung, Seoul (KR); Youn Woong Choi, Gyeonggi-do (KR); Sang Min Cho, Gyeonggi-do (KR); Dae Chul Ha, Chungeheongnam-do (KR); Do Hyung Kim, Seoul (KR); Seong Woo Ahn, Seoul (KR); Jeong Hyun Choi, Seoul (KR)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignees: Hannam University Institute dor Industry- Academia Corporation; Korea Umited Pharm, Inc.

(21) Appl. No.: 12/597,451

(22) PCT Filed: Apr. 22, 2008
(86) PCT No.: PCT/KR08/02257

§ 371 Date: Mar. 23, 2010

(30) Foreign Application Priority Data
Apr. 27, 2007 (KR) 10-200700041380
Oct. 31, 2007 (KR) 10-2007-0110502

(51) Int. Cl. A61K 9/14; A61K 31/337; A61P 35/00

(52) U.S. Cl. 424/501; 514/449

(57) ABSTRACT

Disclosed are biocompatible polymeric nanoparticles for drug delivery and a method for preparing the same. They can be prepared by mixing a tri-block copolymer, PEG, and a drug at a predetermined temperature to give a homogeneous polymeric mixture; solidifying the homogeneous polymeric mixture at room temperature; and dissolving the solidified polymeric mixture in an aqueous solution. Based on a polymer melting process, the method makes it easy to produce poloxamer nanoparticles at low cost. The nanoparticles show desired particle sizes suitable for use in drug delivery and a uniform particle size distribution. Consisting of a bilayer structure, the nanoparticles can contain sparingly soluble drugs. Also, the nanoparticles contain no organic solvents and are thus safe to the body because they are free of organic solvent residuals. Further, after being administered into the body, the nanoparticles with a high content of sparingly soluble drug entrapped therein can safely deliver the drug to target sites and stably release the drug at a controlled rate.

### TECHNICAL FIELD

The present invention relates to a method for preparing biocompatible polymeric nanoparticles for use in a drug delivery system based on a polymer melting process. More particularly, the present invention relates to a method for the preparation of biocompatible polymeric nanoparticles for drug delivery by mixing a tri-block copolymer, Polyethylene glycol (PEG), and a drug at a predetermined temperature to yield a homogeneous polymeric mixture, solidifying the homogeneous polymeric mixture at room temperature, and dissolving the solidified polymeric mixture in an aqueous solution. Also, the present invention is concerned with biocompatible polymeric nanoparticles with a sparingly soluble drug entrapped therein, prepared by the method, which can release the drug at target sites in the body.

### BACKGROUND ART

With the great advances in pharmaceutics, various new high-performance drugs have been developed. Many of the newly developed drugs, however, are highly limited in their clinical usefulness due to the very poor solubility thereof.

In order to overcome this problem, active research into hydrotropic polymeric micelles based on copolymers has been conducted (Journal of Controlled Release, 2004, Volume 97, Number 2, pp 249-257, by Yong Woo Cho et al., Journal of Drug Target, 2005, Volume 13, Number 1, pp. 73-80, by Junping Wang et al.).

Attempts have been made to use stable polymeric micelle compositions in solubilizing Paclitaxel, a sparingly soluble anticancer agent (Korean Patent No. 421, 451, and Japanese Patent Laid-Open Publication No. 1990-335267).

In the previous articles and patents, polymeric micelles formed of block copolymers consisting of hydrophilic segments and hydrophobic segments are employed as drug carriers.

Most of the previously documented block copolymers have, however, not been deemed safe for use in the body, and entail many problems upon clinical application.

### DISCLOSURE

### Technical Problem

Accordingly, the present invention has been made keeping in mind the above problems occurring in the prior art, and an object of the present invention is to provide a method for preparing biocompatible polymeric nanoparticles, based on biocompatible polymers safe to the body, which can contain a high load of sparingly soluble drugs and can release the drugs at controlled rates.

It is another object of the present invention to provide biocompatible polymeric nanoparticles, based on biocompatible polymers safe to the body, which can contain a high load of sparingly soluble drugs and release the drugs at controlled rates.

It is a further object of the present invention to provide a method for preparing biocompatible polymeric nanoparticle aggregates which can contain a high load of sparingly soluble drugs and release the drugs at controlled rates.

It is still a further object of the present invention to provide biocompatible polymeric nanoparticle aggregates which can contain a high load of sparingly soluble drugs and release the drugs at controlled rates.

It is still another object of the present invention to provide a method for the preparation of biocompatible polymeric nanoparticles, which is friendly to the environment and to the body.

### Technical Solution

In order to accomplish the above objects, the present invention provides a method for preparing biocompatible polymeric nanoparticles for drug delivery, comprising: mixing a tri-block copolymer, a polyethylene glycol (PEG), and a drug at a predetermined temperature to give a homogeneous polymeric mixture; solidifying the homogeneous polymeric mixture at room temperature; and dissolving the solidified polymeric mixture in an aqueous solution.

Further, the present invention provides a method for preparing biocompatible polymeric nanoparticles for drug delivery, comprising: mixing a tri-block copolymer, a polyethylene glycol (PEG), and a drug at a predetermined temperature to give a homogeneous polymeric mixture; solidifying the homogeneous polymeric mixture at room temperature; dissolving the solidified polymeric mixture in an aqueous solution and freeze-drying the dissolved polymeric mixture to form a tri-block polymer bilayer; and dissolving the tri-block polymer bilayer in an aqueous solution.

Also, the present invention provides a method for the preparation of biocompatible polymeric nanoparticles for drug delivery, comprising: mixing a tri-block copolymer, a polyethylene glycol (PEG), and a drug at a predetermined temperature to give a homogeneous polymeric mixture; solidifying the homogeneous polymeric mixture at a low temperature; and dissolving the solidified polymeric mixture in an aqueous solution.

Also, the present invention provides biocompatible polymeric nanoparticles for drug delivery, prepared using the method.

The above objects could be accomplished by providing a method for preparing biocompatible polymeric nanoparticle aggregates for drug delivery, comprising: mixing a tri-block copolymer, a polyethylene glycol (PEG), and a drug at a predetermined temperature to give a homogeneous polymeric mixture; and cooling and solidifying the homogeneous polymeric mixture.

Also provided are biocompatible polymeric nanoparticles for drug delivery prepared according to this method.

### ADVANTAGEOUS EFFECTS

Based on a polymer melting process, as described above, the method for the preparation of biocompatible polymeric nanoparticles for drug delivery in accordance with the present invention is useful for easily producing poloxamer nanoparticles at low cost. The poloxamer nanoparticles prepared using the method show desired particle sizes suitable for use in drug delivery and a uniform particle size distribution. Consisting of a bilayer structure, the poloxamer nanoparticles of the present invention can contain sparingly soluble drugs. Also, the poloxamer nanoparticles contain no organic solvents and are thus safe for use in the body because they are free of organic solvent residuals. Further, after being administered in the body, the poloxamer nanoparticles of the present invention, with a high content of sparingly soluble drug entrapped therein, can safely deliver the drug to target sites and can stably release the drug at a controlled rate.

### DESCRIPTION OF DRAWINGS

FIG. 1 is a histogram showing the particle size distribution of the nanoparticles prepared according to the present invention;

FIG. 2 is a Cryo-TEM (transmittance electron microscopy) photograph showing biocompatible polymeric nanoparticles for drug delivery, prepared according to the present invention;

FIG. 3 is a graph showing the Paclitaxel release pattern of the nanoparticles prepared according to the present invention;

FIG. 4 is a graph showing the Docetaxel release pattern of the nanoparticles prepared according to the present invention; and

FIG. 5 is an FE-SEM (field emission scanning electron microscopy) photograph showing the biocompatible polymeric nanoparticle aggregates for drug delivery prepared according to the present invention.

### BEST MODE

In accordance with an aspect thereof, the present invention pertains to a method for the preparation of biocompatible polymeric nanoparticles for drug delivery on the basis of a polymer melting process, by melting poloxamer, polyethylene glycol and a sparingly soluble drug together at a high temperature to give a viscous molten mixture, cooling the viscous molten mixture to give a solid mixture, and dissolving the mixture in distilled water.

In greater detail, the biocompatible polymeric nanoparticles for drug delivery according to the present invention can be prepared using a method comprising mixing a tri-block copolymer represented by the following Chemical Formula 1, a polyethylene glycol (PEG), represented by the following Chemical Formula 2, and a drug at a predetermined temperature to give a homogeneous polymeric mixture; solidifying the homogeneous polymeric mixture at room temperature; and dissolving the solidified polymeric mixture in an aqueous solution.

[Chemical Formula 1]

HO(C₂H₄O)ₐ(C₃H₆O)_{b}(C₂H₄O)_{c}H

wherein b is an integer of 10 or higher, and a sum of a and c is set such that the terminal moieties corresponding thereto amount to 5-95% by weight, based on the total weight of the polymer, and preferably 20-90% by weight.

[Chemical Formula 2]

HO(C₂H₄O)ₐH

wherein a is an integer of 3 to 1,000.

Based on a polymer melting process, the present invention also pertains to a method for the preparation of biocompatible polymeric nanoparticles for drug delivery, comprising mixing a tri-block copolymer of Chemical Formula 1, a polyethylene glycol (PEG) of Chemical Formula 2, and a drug at a predetermined temperature to give a homogeneous polymeric mixture; solidifying the homogeneous polymeric mixture at room temperature; dissolving the solidified polymeric mixture in an aqueous solution and freeze-drying the dissolved polymeric mixture to form a tri-block polymer bilayer; and dissolving the tri-block polymer bilayer in an aqueous solution.

The tri-block copolymer of Chemical Formula 1 useful in the present invention is polyoxyethylene-polyoxypropylene-polyoxyethylene, named poloxamer, which is soluble in water.

Poloxamer may be prepared according to a method that is well-known in the art, or may be commercially available. The poloxamer useful in the present invention ranges in molecular weight from 1,000 to 16,000 and the property thereof is dependent on the ratio of the hydrophobic polyoxypropylene block to the hydrophilic polyoxyethylene block, that is, the ratio of b to a+c in Chemical Formula 1.

Poloxamer is in a solid state at room temperature and is soluble in water and ethanol. For the generic term “poloxamer”, these copolymers are commonly named with the letter “P” (for poloxamer) followed by digits. Commercially available are P68, 127, 188, 237, 338 and 407. P188 means a poloxamer with a molecular weight of approximately 8,350, in which b is 30 and the sum of a and c is approximately 75.

Polyethylene (PEG), represented by Chemical Formula 2, is an amphipathic polymer exhibiting both hydrophilicity and hydrophobicity. Polyethylene glycol changes in the physical state thereof from a liquid to a solid as the molecular weight increases. As in commercially available PEGs, such as PEG 150, 300, 400, 1000, 6000, 8000, 10000, 20000, 30000 and 40000, the numbers that are often included in the names of PEGs indicate their average molecular weights. For example, PEG 300 would have an average molecular weight of approximately 300 daltons. Particularly, polyethylene glycol having a molecular weight greater than 10000 daltons is called polyethylene oxide (PEO).

Of them, PEG400 is in a liquid state and is often used to solubilize various sparingly soluble drugs. Further, it has received approval from the FDA for use in intravenous injection to the human body.

In accordance with the present invention, the tri-block copolymer is mixed with polyethylene glycol at a ratio of 2:8 to 99:1, and preferably at a ratio of 5:5 to 9:1. When the ratio of the tri-block copolymer (poloxamer) to polyethylene glycol (PEG) falls outside this range, nanoparticles may be obtained at a poor yield, or drug release may sharply increase.

The temperature at which poloxamer, PEG, and a drug melt in accordance with the present invention ranges from 40 to 70° C., and preferably from 50 to 60° C. Heating the poloxamer, PEG and sparingly soluble drug together produces a polymeric mixture as a homogenous viscous liquid.

Next, when the homogenous viscous liquid of the polymeric mixture is cooled, it is solidified to form a structure in which the drug is soluble within the polyethylene glycol inside the poloxamer. The solidified structure is then suspended in an aqueous solution to obtain nanoparticles with the drug entrapped therein.

The solidification of the homogenous polymeric mixture may be conducted by leaving the polymeric mixture at room temperature or by cooling in a temperature-controllable reactor at a controlled rate.

Herein, the term “room temperature” is intended to refer to an ambient temperature of 15° C. or higher.

Particular limitations are not imposed on the cooling rate and temperature for the viscous liquid. Generally, the cooling rate when the viscous liquid is allowed to stand at room temperature is sufficient to achieve solidification. If necessary, a cooling condenser or a temperature-controllable reactor may be used to cool the viscous liquid at a controlled rate.

In the present invention, it generally takes 10 min-1 hr to dissolve the solidified mixture to form nanoparticles. However, the time period may vary depending on the content of the solidified mixture.

In accordance with a further aspect, the present invention pertains to biocompatible polymeric nanoparticles for drug delivery, prepared by the method of the present invention.

The biocompatible polymeric nanoparticles for drug delivery are poloxamer particles which are capable of entrapping a great amount of sparingly soluble drugs therein and the drug release behavior of which can be freely controlled.

The biocompatible polymeric nanoparticles of the present invention range in mean size from 100 nm to 10 μm, and preferably from 50 nm to 5 μm and the most preferably from 10 nm to 3 μm.

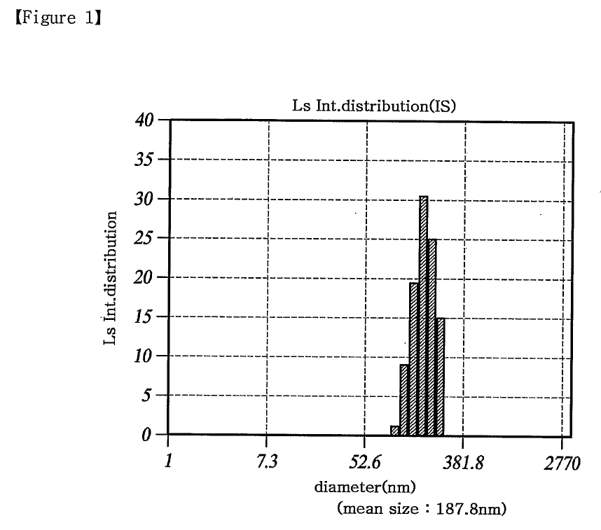

With reference to FIG. 1, the biocompatible polymeric nanoparticles are found to show a uniform particle size distribution, as measured by a particle size analyzer.

In the nanoparticles are contained drugs or biologically active agents. In the case where the molten mixture of poloxamer and polyethylene glycol contains drugs or biologically active agents, most of them are entrapped within microcapsules of poloxamer at a high yield. No particular limitations are imposed on the drugs or biologically active agents useful in the present invention, with the exception that they are substantially stable at around 55° C.

According to the present invention, nanoparticles can be prepared at low cost to have a desired particle sizes within a desired particle size distribution, with various drugs and biologically active agents loaded therein.

As described above, the nanoparticles of the present invention contain no organic solvents. The absence of organic solvents in the preparation of the poloxamer nanoparticles ensures that no organic residuals are produced, thus ensuring safety.

In accordance with still a further aspect thereof, the present invention pertains to a method for the preparation of biocompatible polymeric nanoparticles for drug delivery, comprising mixing a tri-block copolymer of Chemical Formula 1, a polyethylene glycol (PEG) of Chemical Formula 2, and a drug at a predetermined temperature to give a homogeneous polymeric mixture; solidifying the homogeneous polymeric mixture at a low temperature; and dissolving the solidified polymeric mixture in an aqueous solution.

The solidification of the homogenous polymeric mixture is conducted at −100 to 15° C.

At such a low temperature, the homogeneous polymeric mixture is rapidly cooled to entrap a great content of the sparingly soluble drug, so that the drug can be released at a controlled rate. Thanks to this rapid cooling process, the biocompatible, synthetic polymeric nanoparticles for drug delivery in accordance with the present invention can be stably produced in a large amount.

In this aspect, the tri-block copolymer of Chemical Formula 1, the polyethylene glycol of Chemical Formula 2, and the mixture ratio of the tri-block copolymer to the polyethylene glycol are the same as described above. Here, for the temperature used in the mixing step, reference may be made to the description above.

Also, the present invention pertains to biocompatible polymeric nanoparticles for drug delivery, which are prepared using the method.

The above description applies to these biocompatible polymeric nanoparticles for drug delivery.

In accordance with still another aspect thereof, the present invention pertains to a method for the preparation of biocompatible polymeric nanoparticle aggregates for drug delivery, comprising mixing a tri-block copolymer of Chemical Formula 1, a polyethylene glycol (PEG) of Chemical Formula 2, and a drug at a predetermined temperature to give a homogeneous polymeric mixture; and cooling and solidifying the homogeneous polymeric mixture.

When the nanoparticle aggregates for drug delivery, prepared by cooling and solidifying the homogeneous polymeric mixture, including a sparingly soluble drug, are administered, the polymeric components except for the nanoparticles are dissolved man aqueous solution with the sparingly soluble drug remaining entrapped in the nanoparticles, thereby releasing the drug at a controlled rate.

After being administered into the body, the nanoparticle aggregates can safely reach a target site with the drug entrapped within the microparticles.

As described above, the nanoparticle aggregates for drug delivery, prepared by the solidification of the homogeneous polymeric mixture through cooling, are a mixture of nanoparticles and polymeric materials. In an aqueous environment, the polymeric materials of the nanoparticle aggregates are dissolved to separate the nanoparticles, followed by the release of the drug from the nanoparticles.

In this aspect, the tri-block copolymer of Chemical Formula 1, the polyethylene glycol of Chemical Formula 2, and the mixture ratio of the tri-block copolymer to the polyethylene glycol are the same as described above. Also, for the temperature used in the mixing step, reference may be made to the above description.

The solidification of the nanoparticle aggregates for drug delivery is conducted at −100 to 50° C.

In accordance with yet another aspect thereof, the present invention pertains to biocompatible polymeric nanoparticle aggregates, prepared by the method based on the polymer melting process.

In the nanoparticle aggregates, a drug or a biologically active agent is entrapped.

No organic solvent is contained in the nanoparticle aggregates.

The nanoparticles of the nanoparticle aggregates show a uniform particle size distribution.

### MODE FOR INVENTION

A better understanding of the present invention may be obtained through the following examples which are set forth to illustrate, but are not to be construed as the limit of the present invention.

### Example 1

0.8 g of poloxamer (polyoxyethylene-polyoxypropylene-polyoxyethylene tri-block copolymer, F-68) and 0.2 g of polyethylene glycol 400 (PEG 400) were introduced into a reactor and heated to 55° C. The mixture was completely melted by heating at that temperature for 20 min. The resulting viscous liquid was allowed to stand at room temperature (25° C.) to form a solid. This was dissolved in distilled water, followed by filtration through a 0.45 μm filter to obtain poloxamer nanoparticles having a mean diameter size of 50˜500 nm.

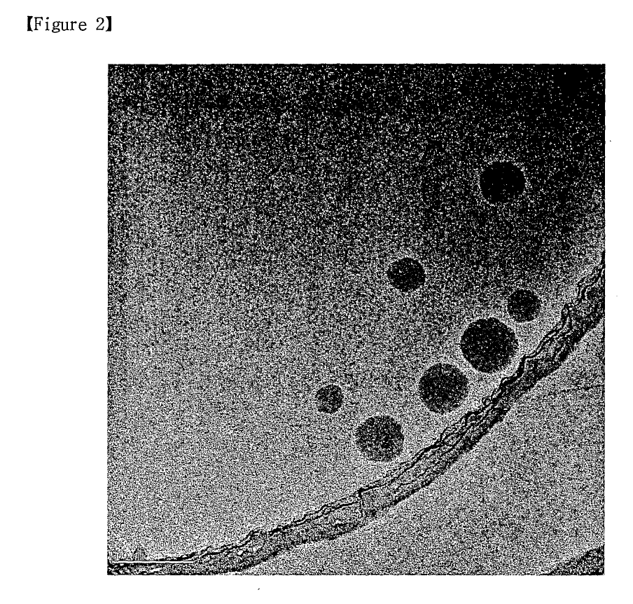

FIG. 2 is a cryo-TEM (transmittance electron microscopy) photograph in which the poloxamer nanoparticles are seen as black crystals.

### Example 2

The same procedure as in Example 1 was repeated, with the exception that, instead of poloxamer (F-68), poloxamer (F-127) having a longer chain of polyoxyethylene was used.

The poloxamer nanoparticles thus obtained were measured to have a mean particle size of 200˜500 nm.

### Example 3

The same procedure as in Example 1 was repeated, with the exception that 0.042 g of the anticancer agent Paclitaxel was used along with the poloxamer.

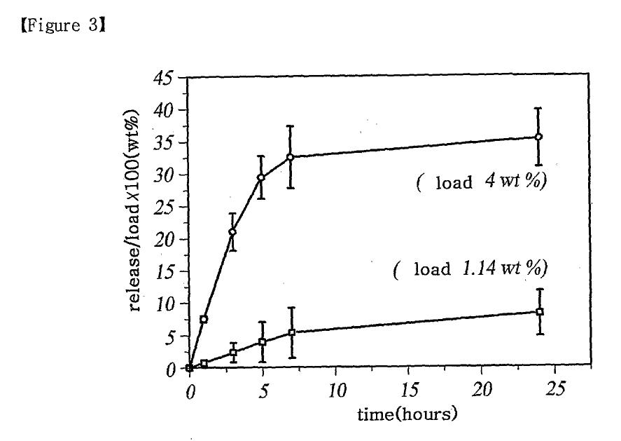

As a result, the poloxamer nanoparticles thus produced entrapped Paclitaxel therein. The poloxamer particles were found to contain paclitaxel at a load of 98% or higher, as measured through high-performance liquid chromatography (HPLC). In FIG. 3, the release pattern of the drug from the nanoparticles is depicted.

### Example 4

The same procedure as in Example 2 was repeated, with the exception that 0.042 g of Docetaxel was used along with the poloxamer.

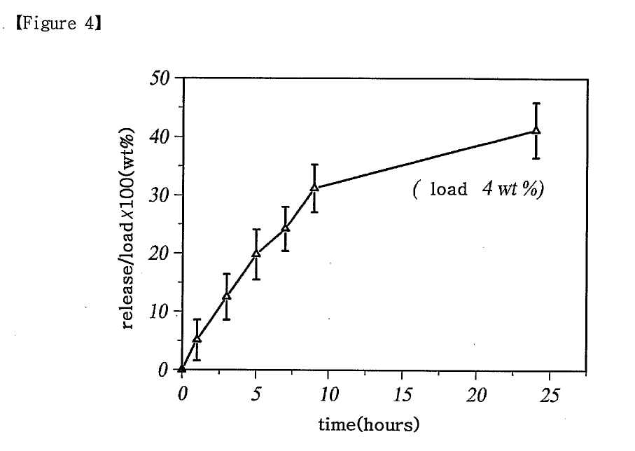

As a result, the poloxamer nanoparticles thus produced entrapped Docetaxel therein. The poloxamer particles were found to contain Docetaxel at a load of 98% or higher, as measured by high-performance liquid chromatography (HPLC). With reference to FIG. 4, the release pattern of the drug from the nanoparticles is depicted.

### Example 5

The same mixture as that solidified in Example 1 was dissolved in 5 ml of a 1 wt % or 5 wt % poloxamer aqueous solution, and then freeze-dried to afford poloxamer nanoparticles having a bilayer structure.

### Example 6

The same procedure as in Example 5 was repeated, with the exception that 0.042 g of the anticancer agent Paclitaxel was used along with the poloxamer.

As a result, the poloxamer nanoparticles thus produced had a bilayer structure with Paclitaxel entrapped therein. The poloxamer particles were found to contain paclitaxel at a load of 98% or higher, as measured through high-performance liquid chromatography (HPLC). With reference to FIG. 3, the release pattern of the drug from the nanoparticles is depicted. As seen in FIG. 3, the drug release was decreased due to the bilayer structure. Accordingly, nanoparticles with desired drug release rates could be prepared in this manner.

### Example 7

The same procedure as in Example 5 was repeated, with the exception that 0.042 g of Docetaxel was used along with the poloxamer.

As a result, the poloxamer nanoparticles thus produced had a bilayer structure with Docetaxel entrapped therein. The poloxamer particles were found to contain Docetaxel at a load of 98% or higher as measured by high-performance liquid chromatography (HPLC).

### Example 8

The same procedure as in Example 3 was repeated, with the exception that the homogenous polymeric mixture was cooled at −70° C.

The poloxamer nanoparticles thus obtained were measured to have a mean particle size of 50˜500 nm.

### Example 9

The procedure of Example 3 was repeated, with the exception that the homogenous polymeric mixture was cooled at 0° C. and the solidified mixture was obtained without being dissolved in distilled water.

The poloxamer nanoparticle aggregates thus obtained were measured to have a mean particle size of 50˜500 nm.

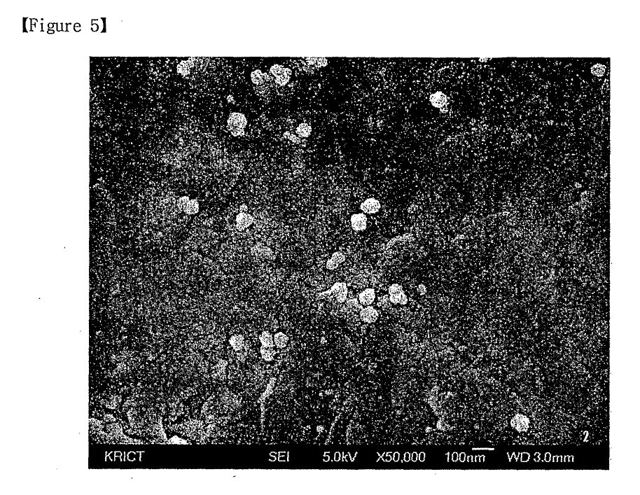

With reference to FIG. 5, the poloxamer nanoparticle aggregates are shown in an FE-SEM (field emission scanning electron microscopy) photograph. In the microphotograph, nanoparticles are visualized as white crystals against the black background for the polymeric materials, indicating that the nanoparticles are not separated from the polymeric materials.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1-21. (canceled)

22. A method for preparing biocompatible polymeric nanoparticles for drug delivery, comprising the steps of:
mixing a tri-block copolymer represented by Chemical Formula 1, a polyethylene glycol (PEG) represented by Chemical Formula 2, and a drug at a predetermined temperature to generate a homogeneous polymeric mixture, wherein:
Chemical Formula 1 is HO(C₂H₄O)ₐ(C₃H₆O)_{b}(C₂H₄O)_{c}H,
wherein in Chemical Formula 1:
‘b’ is an integer of 10 or higher, and
a sum of ‘a’ and ‘c’ is set such that the terminal moieties corresponding thereto amount to 5-95% by weight, based on the total weight of the entire polymer; and
Chemical Formula 2 is HO(C₂H₄O)ₐH,
wherein in Chemical Formula 2:
‘a’ is an integer of 3 to 1,000;
solidifying the homogeneous polymeric mixture at room temperature to generate a first solid;
optionally dissolving the first solid in an aqueous solution to generate an intermediate solution, and freeze-drying the intermediate solution to generate a second solid;
dissolving the first or the second solid in an aqueous solution, to generate a solution of the polymeric nanoparticles.

23. The method of claim 22, wherein the terminal moieties of Chemical Formula 1 amount to 20-90% by weight, based on the total weight of the entire polymer of Chemical Formula 1.

24. The method of claim 22, wherein the tri-block copolymer has a structure of polyoxyethylene-polyoxypropylene-polyoxyethylene and is water-soluble.

25. The method of claim 24, wherein the tri-block copolymer is poloxamer.

26. The method of claim 22, wherein the polyethylene glycol of Chemical Formula 2 is an amphipathic molecule exhibiting both hydrophilicity and hydrophobicity.

27. The method of claim 22, wherein the tri-block copolymer is mixed with the polyethylene glycol at a mixture ratio ranging from 2:8 to 99:1.

28. The method of claim 28, wherein the tri-block copolymer is mixed with the polyethylene glycol at a mixture ratio ranging from 5:5 to 9:1.

29. The method of claim 22, wherein the mixing is conducted at a temperature ranging from 40 to 70° C.

30. The method of claim 29, wherein the mixing is conducted at a temperature ranging from 50 to 60° C.

31. The method of claim 22, wherein the solidifying is conducted by leaving the homogenous polymeric mixture at room temperature or by cooling the homogenous polymeric mixture with a temperature-controllable reactor at a controlled cooling rate.

32. The method of claim 22, wherein the homogenous polymeric mixture is cooled at a temperature ranging from −100 to 15° C.

33. A method for preparing biocompatible polymeric nanoparticle aggregates for drug delivery, comprising the steps of:
mixing a tri-block copolymer of Chemical Formula 1, a polyethylene glycol (PEG) of Chemical Formula 2, and a drug at a predetermined temperature to give a homogeneous polymeric mixture, wherein
Chemical Formula 1 is HO(C₂H₄O)ₐ(C₃H₆O)_{b}(C₂H₄O)_{c}H,
wherein in Chemical Formula 1:
‘b’ is an integer of 10 or higher, and
a sum of ‘a’ and ‘c’ is set such that the terminal moieties corresponding thereto amount to 5-95% by weight, based on the total weight of the entire polymer;
Chemical Formula 2 is HO(C₂H₄O)ₐH,
wherein in Chemical Formula 2:
‘a’ is an integer of 3 to 1,000; and
cooling and solidifying the homogeneous polymeric mixture to generate the nanoparticle aggregates.

34. A composition comprising biocompatible polymeric nanoparticles for drug delivery, wherein the nanoparticles are prepared by the method comprising the steps of:
mixing a tri-block copolymer represented by Chemical Formula 1, a polyethylene glycol (PEG) represented by Chemical Formula 2, and a drug at a predetermined temperature to give a homogeneous polymeric mixture, wherein
Chemical Formula 1 is HO(C₂H₄O)ₐ(C₃H₆O)_{b}(C₂H₄O)_{c}H,
wherein in Chemical Formula 1:
‘b’ is an integer of 10 or higher, and
a sum of ‘a’ and ‘c’ is set such that the terminal moieties corresponding thereto amount to 5-95% by weight, based on the total weight of the entire polymer; and
Chemical Formula 2 is HO(C₂H₄O)ₐH,
wherein in Chemical Formula 2:
‘a’ is an integer of 3 to 1,000;
solidifying the homogeneous polymeric mixture at room temperature to generate a solidified polymeric mixture; and
dissolving the solidified polymeric mixture in an aqueous solution to generate a solution of the biocompatible polymeric nanoparticles.

35. The composition of claim 34, wherein the biocompatible polymeric nanoparticles range in mean particle size from 100 nm to 10 μm.

36. The composition of claim 34, wherein the biocompatible polymeric nanoparticles range in mean particle size from 50 nm to 5 μM.

37. The composition of claim 34, wherein the biocompatible polymeric nanoparticles range in mean particle size from 10 nm to 3 μM.

38. The composition of claim 34, wherein the biocompatible polymeric nanoparticles have a uniform particle size distribution.

39. The composition of claim 34, wherein the biocompatible polymeric nanoparticles contain a drug or a biologically active agent therein.

40. The composition of claim 34, wherein the biocompatible polymeric nanoparticles contain no organic solvents.

41. A composition comprising biocompatible polymeric nanoparticles for drug delivery, wherein the nanoparticles are prepared by the method comprising the steps of:
mixing a tri-block copolymer of Chemical Formula 1, a polyethylene glycol (PEG) of Chemical Formula 2, and a drug at a predetermined temperature to give a homogeneous polymeric mixture, wherein
Chemical Formula 1 is HO(C₂H₄O)ₐ(C₃H₆O)_{b}(C₂H₄O)_{c}H,
wherein in Chemical Formula 1:
‘b’ is an integer of 10 or higher, and
a sum of ‘a’ and ‘c’ is set such that the terminal moieties corresponding thereto amount to 5-95% by weight, based on the total weight of the entire polymer; and
Chemical Formula 2 is HO(C₂H₄O)ₐH,
wherein in Chemical Formula 2:
‘a’ is an integer of 3 to 1,000; and
cooling and solidifying the homogeneous polymeric mixture to generate the biocompatible polymeric nanoparticles.
